(19) United States
(12) Patent Application Publication
(10) Pub. No.: US 2023/0293556 A1
(43) Pub. Date: Sep. 21, 2023

(54) PHARMACEUTICAL COMPOSITION OF AQUAPORIN INHIBITOR AND PREPARATION METHOD THEREOF

(71) Applicants: JIANGSU SIMCERE PHARMACEUTICAL CO., LTD., Nanjing (CN); AEROMICS, INC., New Haven, CT (US)

(72) Inventors: Yong QIAN, Nanjing (CN); Ruiwen SHI, Nanjing (CN); Amy LIU, Nanjing (CN); Tian SONG, Nanjing (CN); Yanhong LIU, Nanjing (CN); Aijuan FENG, Nanjing (CN); Jinsheng REN, Nanjing (CN)

(21) Appl. No.: 18/040,618

(22) PCT Filed: Aug. 4, 2021
(86) PCT No.: PCT/CN2021/110506

§ 371 Date: Feb. 3, 2023

(30) Foreign Application Priority Data
Aug. 5, 2020 (CN) 202010779964.6

(51) Int. Cl. A61K 31/661; A61K 31/133; A61K 9/19; A61K 47/26; A61K 47/02; A61K 47/12; A61K 9/00

(52) U.S. Cl. CPC A61K 31/661; A61K 31/133; A61K 9/19; A61K 47/26; A61K 47/02; A61K 47/12; A61K 9/0019

(57) ABSTRACT

Provided are a pharmaceutical composition of an aquaporin inhibitor and a preparation method thereof. The pharmaceutical composition comprises 2-((3,5-bis (trifluoromethyl) phenyl)carbamoyl)-4-chlorophenyl dihydrogen phosphate or a pharmaceutically acceptable salt thereof, or a pharmaceutically acceptable solvate thereof, and meglumine. The pharmaceutical composition of the aquaporin inhibitor and the preparation method thereof have the following advantages: the process is simple, has strong operability, and is conducive to industrial production, and the product has good stability, and obviously less content of degradable impurities, which ensures the effectiveness of the medicine.

### FIELD OF THE INVENTION

This application relates to the field of pharmaceutical compositions, in particular to a pharmaceutical composition of an aquaporin inhibitor and a preparation method thereof.

### BACKGROUND OF THE INVENTION

Aquaporins are cell membrane proteins that act as molecular water channels to mediate the flow of water in and out of the cells. While there is some degree of passive diffusion or osmosis of water across cell membranes, the rapid and selective transport of water in and out of cells involves aquaporins. These water channels selectively conduct water molecules in and out of the cell, while blocking the passage of ions and other solutes, thereby preserving the membrane potential of the cell. Aquaporins are found in virtually all life forms, from bacteria to plants to animals. In humans, they are found in cells throughout the body.

Aquaporin inhibitors, e.g., inhibitors of AQP4 and/or AQP2, may be of utility in the treatment or control of diseases of water imbalance, for example edema (particularly edema of the brain and spinal cord), hyponatremia, and excess fluid retention, as well as diseases such as epilepsy, retinal ischemia and other diseases of the eye, myocardial ischemia, myocardial ischemia/reperfusion injury, myocardial infarction, myocardial hypoxia, congestive heart failure, sepsis, and neuromyelitis optica, as well as migraines.

WO2013169939 discloses N-[3,5-bis(trifluoromethyl)phenyl]-5-chloro-2-hydroxy-benzamide (structure shown in formula (II)) as an aquaporin inhibitor. Formula (I) (structure shown below) is a prodrug of formula (II). The compounds can treat or control aquaporin-mediated diseases selected from cytotoxic brain edema, spinal cord edema, retinal edema, optic nerve edema, cardiac edema, optic neuromyelitis, hyponatremia, retinal ischemia, and excessive fluid retention.

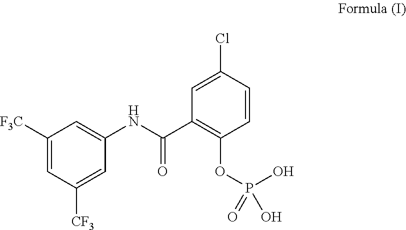 [SMILES: O=C(Nc1cc(C(F)(F)F)cc(C(F)(F)F)c1)c1cc(Cl)ccc1OP(=O)(O)O]

The formula (I) compound needs to be prepared as a liquid formulation for intravenous injection or infusion to achieve rapid onset of action. However, the aqueous solubility of formula (I) needs to be improved to allow for an injection that provides a therapeutically effective amount of formula (II). Yet, salts of formula (I), which are more soluble, can revert to N-[3,5-bis(trifluoromethyl)phenyl]-5-chloro-2-hydroxybenzamide (formula (II) compound) even in the solid state. WO2015069956 shows that certain lyophilized salts of formula (I) revert to formula (II) even in the solid state (about 1% per day or 1% in 5 days). There is an urgent need to solve the problem of drug solubility and stability in order to provide a pharmaceutical composition that fulfills the requirements of clinical medication.

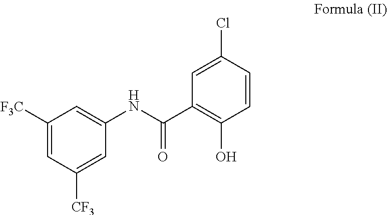 [SMILES: O=C(Nc1cc(C(F)(F)F)cc(C(F)(F)F)c1)c1cc(Cl)ccc1O]

### SUMMARY OF THE INVENTION

The application provides a pharmaceutical composition, which comprises 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine (also called N-methyl-D-glucamine).

In some embodiments, the weight ratio of 2-((3,5-bis(trifluoromethyl)phenyl)carbamoyl)-4-chlorophenyl dihydrogen phosphate to meglumine is 1:0.2˜4. In some embodiments, the weight ratio of 2-((3,5-bis(trifluoromethyl)phenyl)carbamoyl)-4-chlorophenyl dihydrogen phosphate to meglumine is 1:0.4˜2. In some embodiments, the weight ratio of 2-((3,5-bis(trifluoromethyl)phenyl)carbamoyl)-4-chlorophenyl dihydrogen phosphate to meglumine is 1:0.6˜1.

In some embodiments, the pharmaceutical composition is an injectable pharmaceutical composition.

In some embodiments, the pharmaceutical composition is a lyophilized pharmaceutical composition.

In some embodiments, the pharmaceutical composition in the present application further comprises a lyophilization excipient.

In some embodiments, the lyophilization excipient is selected from one or a mixture of sucrose, lactose, mannitol, glucose, and trehalose. In some embodiments, the lyophilization excipient is selected from one or a mixture of sucrose, lactose, and trehalose.

In some embodiments, the weight ratio of 2-((3,5-bis(trifluoromethyl)phenyl)carbamoyl)-4-chlorophenyl dihydrogen phosphate to lyophilization excipient is 1:1˜10. In some embodiments, the weight ratio of 2-((3,5-bis(trifluoromethyl)phenyl)carbamoyl)-4-chlorophenyl dihydrogen phosphate to lyophilization excipient is 1:2.5˜7.5. In some embodiments, the weight ratio of 2-((3,5-bis(trifluoromethyl)phenyl)carbamoyl)-4-chlorophenyl dihydrogen phosphate to lyophilization excipient is 1:5.

Optionally, the pharmaceutical composition in the present application further comprises a pH adjusting agent. The pH adjusting agent in the present application is selected from one or a mixture of hydrochloric acid, sodium hydroxide, citric acid, and phosphate buffer. In some embodiments, the pH adjusting agent in the present application is selected from hydrochloric acid and citric acid.

In some embodiments, the pH of the pharmaceutical composition in the present application is 7.5 to 9.5. In some embodiments, the pH of the pharmaceutical composition in the present application is 8.0 to 9.0. In some embodiments, the pH of the pharmaceutical composition in the present application is about 8.5.

In some embodiments, the application provides a pharmaceutical composition, which comprises 2-((3,5-bis(trifluoromethyl)phenyl)carbamoyl)-4-chlorophenyl dihydrogen phosphate or a pharmaceutically acceptable salt thereof, or a pharmaceutically acceptable solvate thereof, meglumine, a lyophilization excipient and a pH adjusting agent.

Optionally, the pharmaceutical composition in the present application further includes water for injection.

In some embodiments, the application provides a method for preparing the above-mentioned pharmaceutical composition, including:
a) Take 60%˜90% of the prescription amount of water for injection, cool to 15° C.˜25° C., add meglumine and dissolve until clear, slowly add 2-((3,5-bis(trifluoromethyl)phenyl)carbamoyl)-4-chlorophenyl dihydrogen phosphate or a pharmaceutically acceptable salt thereof, or a pharmaceutically acceptable solvate thereof.
b) Optionally, add other components;
c) Add prescribed amount of water for injection;
d) Optionally, filter sterilize and lyophilize the product obtained in step (c).

In some embodiments, step (a) is cooled to 20° C.

In some embodiments, the other components in step (b) are selected from lyophilization excipients and pH adjusting agents.

In some embodiments, the lyophilizing process in step (d) is as follows: (1) preserve at −50° C. for 2˜6 h; (2) raise temperature to −20° C. to −10° C. and preserve for 20˜40 h; (3) raise temperature to 20˜30° C. and preserve for 10˜30 h. In some embodiments, the temperature is raised to 25° C. in step (3).

In some embodiments, the lyophilizing process in step (d) is as follows: (1) decrease temperature to −50° C.; (2) increase temperature to −15° C.; (3) decrease temperature to −50° C.; (4) apply vacuum; (5) decrease temperature to −10° C.; (6) raise temperature to −5° C.; and (7) raise temperature to 25° C. under vacuum.

In some embodiments, the lyophilizing process in step (d) is as follows: (1) decrease temperature (e.g., shelf temperature) to −40° C. to −60° C. (e.g., −50° C.) within 2-6 hours (e.g., within 4 hours); (2) maintain the temperature (e.g., shelf temperature) at −40° C. to −60° C. (e.g., −50° C.) for 1-2 hours (e.g., 0.5 hours); (3) increase (e.g., rapidly) temperature (e.g., shelf temperature) to −20° C. to −10° C. (e.g., −15° C.); (4) maintain the temperature at −20° C. to −10° C. for 1-3 hours (e.g., 2 hours); (5) decrease (e.g., rapidly) temperature (e.g., slab temperature) to −40° C. to −60° C. (e.g., −50° C.); (6) maintain the temperature (e.g., slab temperature) at −40° C. to −60° C. (e.g., −50° C.) for 2-6 hours (e.g., 4 hours); (7) apply vacuum (e.g., to achieve vacuum below 0.2 mbar); (8) decrease temperature (e.g., shelf temperature) to −20° C. to 0° C. (e.g., −10° C.) within 6-10 hours (e.g., 8 hours); (9) maintain the temperature (e.g., shelf temperature) at −20° C. to 0° C. (e.g., −10° C.) for 8-12 hours (e.g., 10 hours); (10) raise temperature to −10° C. to 0° C. (e.g., −5° C.) within 1-3 hours (e.g., 2 hours); (11) maintain the temperature (e.g., shelf temperature) at −10° C. to 0° C. (e.g., −5° C.) for 13-17 hours (e.g., 15 hours); (12) raise temperature (e.g., shelf temperature) to 20° C.-30° C. (e.g., 25° C.) under vacuum (e.g., ultimate vacuum) within 4-8 hours (e.g., 6 hours); and (13) maintain temperature 20° C.-30° C. (e.g., 25° C.) for 10-14 hours (e.g., 12 hours).

In some embodiments, the lyophilized formulation disclosed herein is reconstituted with an aqueous solution comprising sodium and/or potassium (e.g., comprising sodium chloride, e.g., 0.9% NaCl or comprising sodium chloride and sodium lactate or sodium acetate (e.g., Lactated Ringer's or Acetated Ringer's)) or comprising potassium chloride (e.g., potassium chloride injection)). Reconstitution with water for injection or glucose may result in visible particles.

In some embodiments, the “pharmaceutically acceptable salt” in the present application is selected from alkali metal salts (such as sodium salt, preferably disodium salt), organic base salt (such as ammonium salt, preferably meglumine salt).

In some embodiments, the “pharmaceutically acceptable solvate” in the present application is hydrate (eg, dihydrate).

In this application, when the pharmaceutical composition is in a solid form (for example, a lyophilized formulation), the pH refers to the pH value of the solution of the solid pharmaceutical composition before lyophilization and/or the pH value after reconstitution.

In this application, unless otherwise specified, the weight of 2-((3,5-bis(trifluoromethyl)phenyl)carbamoyl)-4-chlorophenyl dihydrogen phosphate or a pharmaceutically acceptable salt thereof, or a pharmaceutically acceptable solvate thereof is by the weight of 2-((3,5-bis(trifluoromethyl)phenyl)carbamoyl)-4-chlorophenyl dihydrogen phosphate.

Sugars (e.g., trehalose) may be provided in hydrate form. In this application, unless otherwise specified, the weight of sugar (e.g., sucrose, lactose, glucose, or trehalose) is by the weight of the sugar in anhydrate form.

The invention provides a formulation that can be reconstituted to a liquid that is acceptable to the human body for intravenous injection or infusion, which can quickly take effect, so that the formula (I) compound can be used for treatment in the field of cerebral edema. At the same time, the composition of formula (I) compound in the present invention and its preparation method are simple, with great operability, which is conducive to industrial production, and the product has good stability, and the content of degradation impurities (such as the formula (II) compound) is significantly less, which ensures exerting of pharmaceutical efficacy. For instance, compared to compositions comprising other bases, such as a sodium salt base or other amine bases (e.g., arginine, lysine, and histidine), specific compositions comprising meglumine disclosed herein show less reversion of formula (I) to formula (II). Without being bound by theory, it is believed that some bases may drive a unimolecular process that results in formula (I) reverting to formula (II). Formula (II) has poor aqueous solubility, thus even small amounts of it in a formulation for injection may result in visible particles, rendering the formulation unusable. With meglumine, impurities in the lyophilized composition are less and the composition is acceptable for injection after reconstitution.

Provided is a pharmaceutical composition (Composition 1a) comprising 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine.

Further provided is a solid lyophilized pharmaceutical composition (Composition 1b ) comprising a meglumine salt of 2-((3,5-bis(trifluoromethyl)phenyl)carbamoyl)-4-chlorophenyl dihydrogen phosphate (formula (I)):

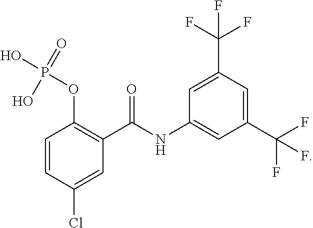 [SMILES: O=C(Nc1cc(C(F)(F)F)cc(C(F)(F)F)c1)c1cc(Cl)ccc1OP(=O)(O)O]

Further provided are Composition 1a and 1b as follows:
1.1. Composition 1a, wherein the composition is a solid lyophilized pharmaceutical composition.
1.2. Any of Compositions 1a, 1b, or 1.1, wherein the pharmaceutically acceptable salt is 2-{[3,5-bis(trifluorornethyl)phenyl]carbamoyl}-4-chlorophenyl phosphate bis-meglumine salt, i.e., wherein the pharmaceutically acceptable salt is:

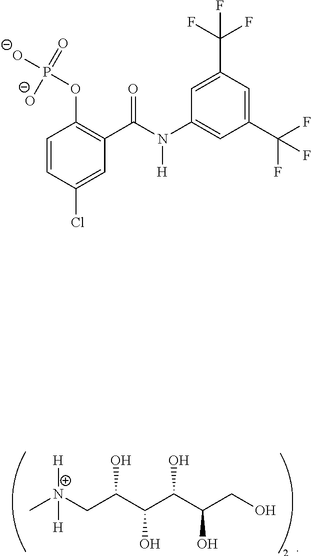

Or, for instance, any of Compositions 1a, 1b, or 1.1, wherein the composition comprises a mixture of 2-((3,5-bis(trifluoromethyl)phenyl)carbamoyl)-4-chlorophenyl dihydrogen phosphate, meglumine, and a meglumine salt of 2((3,5-bis(trifluoromethyl)phenyl)carbamoyl)-4-chlorophenyl dihydrogen phosphate (e.g., 2-{[3,5-bis(trifluoromethyl)phenyl]carbamoyl}-4-chlorophenyl phosphate bis-meglumine salt).
1.3. Any of Compositions 1a, 1b, 1.1, or 1.2, wherein the weight ratio of 2-((3,5-bis(trifluoromethyl)phenyl)carbamoyl)-4-chlorophenyl dihydrogen phosphate (formula (I)) to meglumine is 1:0.2-4, e.g., 1:0.4-2, e.g., 1:0.6-1. Or, for instance, any of Compositions 1a, 1b, 1.1, or 1.2, wherein the amount of meglumine is sufficient to provide a pH of the dissolved composition before and/or after lyophilization of 7 to 10, e.g., 7.5 to 9.5, e.g., 8 to 9, e.g., 8.2-9, e.g., 8.5-8.6, e.g., 8.5.
1.4. Any of Compositions 1a, 1b, or 1.1-1.3, wherein the molar ratio of 2-((3,5-bis(trifluoromethyl)phenyl)carbamoyl)-4-chlorophenyl dihydrogen phosphate (formula (I)) to meglumine is 1:1-5, e.g., 1:2-5, e.g., 1:2-4, e.g., 1:2-3, e.g., 1:2-2.5, e.g., 1:2-2.3 (e.g., 1:2.1-2.3), e.g., 1:2-2.2, e.g., 1:2.1-2.2, e.g., 1:2.2.
1.5. Any of Compositions 1a, 1b, or 1.1-1.3, wherein the molar ratio of deprotonated 2-((3,5-bis(trifluoromethyl)phenyl)carbamoyl)-4-chlorophenyl dihydrogen phosphate (formula (I)) to protonated meglumine is 1:1-5, e.g., 1:2-5, e.g., 1:2-4, e.g., 1:2-3, e.g., 1:2-2.5, e.g., 1:2-2.3 (e.g., 1:2.1-2.3), e.g., 1:2-2.2, e.g., 1:2.1-2.2, e.g., 1:2.2.
1.6. Any of Compositions 1a, 1b, or 1.1-1.5, wherein the composition is for injection after reconstitution, e.g., for intravenous injection and/or infusion.
1.7. Any of Compositions 1a, 1b, or 1.1-1.6, wherein the composition further comprises a lyophilization excipient. For instance, any of Compositions 1a, 1b, or 1.1-1.6, wherein the lyophilization excipient is one or a mixture of a monosaccharide, a disaccharide, and a sugar alcohol. For instance, any of Compositions 1a, 1b, or 1.1-1.6, wherein the lyophilization excipient is a disaccharide or a mixture of disaccharides.
1.8. Composition 1.7, wherein the lyophilization excipient is selected from one or a mixture of sucrose, lactose, mannitol, glucose, and trehalose, any in free or hydrate form. For instance, Composition 1.7, wherein the lyophilization excipient is trehalose, in free or hydrate (e.g., dihydrate) form. For instance, Composition 1.7, wherein the lyophilization excipient is mannitol. For instance, Composition 1.7, wherein the lyophilization excipient is sucrose. For instance, Composition 1.7, wherein the lyophilization excipient is lactose, in free or hydrate (e.g., monohydrate) form.
1.9. Composition 1.8, wherein the lyophilization excipient is selected from one or a mixture of sucrose, lactose, and trehalose.
1.10. Composition 1.8, wherein the lyophilization excipient is sucrose.
1.11. Any of Compositions 1a, 1b, or 1.1-1.10, wherein the weight ratio of 2-((3,5-bis(trifluoromethyl)phenyl)carbamoyl)-4-chlorophenyl dihydrogen phosphate (formula (I)) to the lyophilization excipient is 1:1-10, e.g., 1:2.5-7.5, e.g., 1:2.5-5, e.g., 1:5.
1.12. Any of Compositions 1a, 1b, or 1.1-1.11, wherein the composition further comprises a pH adjusting agent.
1.13. Composition 1.12, wherein the pH adjusting agent is selected from one or a mixture of hydrochloric acid, sodium hydroxide, citric acid, and phosphate buffer.
1.14. Composition 1.12, wherein the pH adjusting agent is selected from hydrochloric acid and citric acid.
1.15. Any of Compositions 1a, 1b, or 1.1-1.14, wherein the pH of the composition is 7 to 10, e.g., 7.5 to 9.5, e.g., 8 to 9, e.g., 8.2-9, e.g., 8.5-8.6, e.g., 8.5.
1.16. Any of Compositions 1a, 1b, or 1.1-1.15, wherein the pH of the composition in solution prior to lyophilization is 7 to 10, e.g., 7.5 to 9.5, e.g., 8 to 9, e.g., 8.2-9, e.g., 8.5-8.6, e.g., 8.5.
1.17. Any of Compositions 1a, 1b, or 1.1-1.16, wherein the pH of the composition after reconstitution is 7 to 10, e.g., 7.5 to 9.5, e.g., 8 to 9, e.g., 8.2-9, e.g., 8.5-8.6, e.g., 8.5.
1.18. Any of Compositions 1a, 1b, or 1.1-1.17, wherein the composition is reconstituted with an aqueous solution comprising sodium and/or potassium (e.g., comprising sodium chloride, e.g., 0.9% sodium chloride injection, or comprising sodium chloride and sodium lactate or sodium acetate, e.g., Lactated Ringer's or Acetated Ringer's, or comprising potassium chloride, e.g., potassium chloride injection).
1.19. Any of Compositions 1a, 1b, or 1.1-1.18, wherein the composition is a white lump. 1.20. Any of Compositions 1a, 1b, or 1.1-1.19, wherein the composition comprises <1% w/w of the compound of formula (II) after 6 months at room temperature (e.g., after 6 months at 25° C.±2° C.), e.g., <0.8% w/w of the compound of formula (II) after 6 months at room temperature (e.g., after 6 months at 25° C.±2° C.), e.g., ≤0.6% w/w of the compound of formula (II) after 6 months at room temperature (e.g., after 6 months at 25° C.±2° C.), e.g., ≤0.5% w/w of the compound of formula (II) after 6 months at room temperature (e.g., after 6 months at 25° C.±2° C.).
1.21. Any of Compositions 1a, 1b, or 1.1-1.20, wherein the composition comprises <2% w/w total impurity (including the compound of formula (II)) after 6 months at room temperature (e.g., after 6 months at 25° C.±2° C.), e.g., ≤1% w/w total impurity after 6 months at room temperature (e.g., after 6 months at 25° C.±2° C.), e.g., ≤0.6% w/w total impurity after 6 months at room temperature (e.g., after 6 months at 25° C.±2° C.), e.g., ≤0.5% w/w total impurity after 6 months at room temperature (e.g., after 6 months at 25° C.±2° C.).
1.22. Any of Compositions 1a, 1b, or 1.1-1.21, wherein the composition comprises ≤1% w/w of the compound of formula (II) after 6 months at 2° C.-8° C., e.g., ≤0.5% w/w of the compound of formula (II) after 6 months at 2° C.-8° C., e.g., ≤0.2% w/w of the compound of formula (II) after 6 months at 2° C.-8° C., e.g., ≤0.1% w/w of the compound of formula (II) after 6 months at 2° C.-8° C.
1.23. Any of Compositions 1a, 1b, or 1.1-1.22, wherein the composition comprises ≤2% w/w total impurity (including the compound of formula (II)) after 6 months at 2° C.-8° C., e.g., ≤0.5% w/w total impurity after 6 months at 2° C.-8° C., e.g., ≤0.2% w/w total impurity after 6 months at 2° C.-8° C., e.g., ≤0.1% w/w total impurity after 6 months at 2° C.-8° C.
1.24. Any of Compositions 1a, 1b, or 1.1-1.23, wherein the composition has <3% w/w water, e.g., ≤2% w/w water, e.g., ≤1.5% w/w water (e.g., after 6 months at 25° C.±2° C. and/or 6 months at 2° C.-8° C.). The amount of water may be may be measured by, for instance, coulometric Karl Fisher analysis.
1.25. Any of Compositions 1a, 1b, or 1.1-1.24, wherein before (e.g., in solution before lyophilization) and/or after reconstitution the composition conforms to USP (U.S. Pharmacopeia) <788> and/or ChP (Pharmacopeia of the People's Republic of China) 2020 Part 4 <0903>.
1.26. Any of Compositions 1a, 1b, or 1.1-1.25, wherein before lyophilization and/or after reconstitution the average number of particles present in the unit(s) tested (e.g., ≤10 units may be tested) does not exceed 25 per mL equal to or greater than 10 μm and does not exceed 3 per mL equal to or greater than 25 μm, e.g., for a preparation (e.g., a parenteral infusion or a solution for injection) supplied in a container with a nominal volume of equal to or more than 100 mL (for instance, for a preparation supplied in a container with a nominal volume of more than 100 mL). The average number of particles present in the unit(s) may be determined by the Light Obscuration Particle Count Test, which is described in USP (U.S. Pharmacopeia) <788> (Light Obscuration Particle Count Test may be performed by, e.g., the AccuSizer® SIS system or HIAC Royco particle counter). The number of unit(s) tested may provide a statistically sound assessment.
1.27. Any of Compositions 1a, 1b, or 1.1-1.26, wherein before lyophilization and/or after reconstitution the average number of particles present in the unit(s) tested (e.g., ≤10 units may be tested) does not exceed 6000 per container equal to or greater than 10 μm and does not exceed 600 per container equal to or greater than 25 μm, e.g., for a preparation (e.g., a parenteral infusion or a solution for injection) supplied in a container with a nominal volume of equal to or less than 100 mL (for instance, for a preparation supplied in a container with a nominal volume of less than 100 mL). The average number of particles present in the unit(s) may be determined by the Light Obscuration Particle Count Test, which is described in USP (U.S. Pharmacopeia) <788> (Light Obscuration Particle Count Test may be performed by, e.g., the AccuSizer® SIS system or HIAC Royco particle counter). The number of unit(s) tested may provide a statistically sound assessment.
1.28. Any of Compositions 1a, 1b, or 1.1-1.27, wherein before lyophilization and/or after reconstitution the average number of particles present in the unit(s) tested (e.g., ≤10 units may be tested) does not exceed 12 per mL equal to or greater than 10 μm and does not exceed 2 per mL equal to or greater than 25 μm, e.g., for a preparation (e.g., a parenteral infusion or a solution for injection) supplied in a container with a nominal volume of equal to or more than 100 mL (for instance, for a preparation supplied in a container with a nominal volume of more than 100 mL). The average number of particles present in the unit(s) may be determined by the Microscopic Particle Count Test, which is described in USP <788>. The number of unit(s) tested may provide a statistically sound assessment.
1.29. Any of Compositions 1a, 1b, or 1.1-1.28, wherein before lyophilization and/or after reconstitution the average number of particles present in the unit(s) tested (e.g., ≤10 units may be tested) does not exceed 3000 per container equal to or greater than 10 μm and does not exceed 300 per container equal to or greater than 25 μm, e.g., for a preparation (e.g., a parenteral infusion or a solution injection) supplied in a container with a nominal volume of equal to or less than 100 mL (for instance, for a preparation supplied in a container with a nominal volume of less than 100 mL). The average number of particles present in the unit(s) may be determined by the Microscopic Particle Count Test, which is described in USP <788>. The number of unit(s) tested may provide a statistically sound assessment.
1.30. Any of Compositions 1a, 1b, or 1.1-1.29, wherein before lyophilization and/or after reconstitution the nephelometric turbidity units (NTU) is <2.2 NTU, e.g., ≤2, e.g., ≤1. NTU may be measured using a nephelometer or a turbidimeter. Any of Compositions a, 1b, or 1.1-1.29, wherein the solution is clear compared to a standard turbidity solution (e.g., a 0.5 standard turbidity solution).
1.31. Any of Compositions 1a, 1b, or 1.1-1.30, wherein the composition is lyophilized from an aqueous solution (e.g., water for injection, e.g., sterile water for injection). Any of Compositions 1a, 1b, or 1.1-1.30, wherein the composition is lyophilized from an aqueous solution substantially free (e.g., containing no added) of an organic solvent (e.g., t-butyl alcohol).
1.32. Any of Compositions 1a, 1b, or 1.1-1.31, wherein the composition before reconstitution is substantially free of Na⁺ and K⁺ ions. For instance, any of Compositions 1a, 1b, or 1.1-1.31, wherein the composition does not contain any detectable Na⁺ or K⁺ ions. For instance, any of Compositions 1a, 1b, or 1.1-1.31, wherein the composition comprises less than 10 ppm (e.g., less than 5 ppm, e.g., less than 1 ppm) of Na⁺ and/or K⁺ ions
1.33. Any of Compositions 1a, 1b, or 1.1-1.32, wherein the composition is not made with a sodium salt (e.g., NaOH, NaH₂PO₄, Na₂HPO₄, or Na₃PO₄) or a potassium salt (e.g., KOH, KH₂PO₄, K₂HPO₄, and K₃PO₄).
1.34. Any of Compositions 1a, 1b, or 1.1-1.33, wherein the composition is substantially free of sodium phosphate (i.e., NaH₂PO₄, Na₂HPO₄, and Na₃PO₄) and potassium phosphate (i.e., KH₂PO₄, K₂HPO₄, and K₃PO₄). For instance, any of Compositions 1a, 1b, or 1.1-1.33, wherein the composition does not contain any detectable sodium phosphate (i.e., NaH₂PO₄, Na₂HPO₄, and Na₃PO₄) or potassium phosphate (i.e., KH₂PO₄, K₂HPO₄, and K₃PO₄).
1.35. Any of Compositions 1a, 1b, or 1.1-1.34, wherein the composition is not made with a sodium phosphate (i.e., NaH₂PO₄, Na₂HPO₄, and Na₃PO₄) or a potassium phosphate (i.e., KH₂PO₄, K₂HPO₄, and K₃PO₄).
1.36. Any of Compositions 1a, 1b, or 1.1-1.35, wherein the composition before and/or after reconstitution is substantially free of a polysaccharide. As used herein, “polysaccharide” means a chain of 10 or more monosaccharide residues linked by glycosidic bonds. For instance, any of Compositions 1a, 1b, or 1.1-1.35, wherein the composition before and/or after reconstitution is substantially free of dextran.
1.37. Any of Compositions 1a, 1b, or 1.1-1.36, wherein the composition before and/or after reconstitution is substantially free of a cyclodextrin (i.e., a cyclic oligosaccharide made up of (α-1,4)-linked α-D-glucopyranoses). For instance, any of Compositions 1a, 1b, or 1.1-1.36, wherein the composition before and/or after reconstitution is substantially free of hydroxypropyl-beta-cyclodextrin or sulfobutylether-P-cyclodextrin (e.g., sulfobutylether-(3-cyclodextrin sodium). For instance, any of Compositions 1a, 1b, or 1.1-1.36, wherein the composition before and/or after reconstitution does not contain any detectable cyclodextrin (e.g., hydroxypropyl-beta-cyclodextrin or sulfobutylether-β-cyclodextrin (e.g., sulfobutylether-β-cyclodextrin sodium)).
1.38. Any of Compositions 1a, 1b, or 1.1-1.37, wherein the composition consists essentially of 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, meglumine, and a monosachharide (e.g., glucose). For instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and a monosaccharide (e.g., glucose). For instance, any of Compositions 1a, 1b, or 1.1-1.37, wherein the composition consists essentially of 2-{[3,5-bis(tri fluoromethyl)phenyl] carbamoyl}-4-chlorophenyl phosphate bis-meglumine salt and glucose. Or, for instance, any of Compositions 1a, 1b, or 1.1-1.37, wherein the composition consists essentially of a mixture of 2-((3,5-bis(trifluoromethyl)phenyl)carbamoyl)-4-chlorophenyl dihydrogen phosphate, meglumine, and a meglumine salt of 2-((3,5-bis(trifluoromethyl)phenyl)carbamoyl)-4-chlorophenyl dihydrogen phosphate (e.g., 2-{[3,5-bis(trifluoromethyl)phenyl]arbamoyl}-4-chlorophenyl phosphate bis-meglumine salt) and a monosaccharide (e.g., glucose).
1.39. Any of Compositions 1a, 1b, or 1.1-1.37, wherein the composition consists essentially of 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, meglumine, and a disachharide (e.g., one or a mixture of sucrose, lactose, or trehalose). For instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and a disaccharide (e.g., one or a mixture of sucrose, lactose, or trehalose). For instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and sucrose. Or, for instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and lactose. Or, for instance, any of Compositions 1a, 1b, or 1.1-1.38, wherein the composition consists essentially of 2-{[3,5-bis(trifluoromethyl)phenyl]carbamoyl}-4-chlorophenyl phosphate bis-meglumine salt and trehalose. Or, for instance, any of Compositions 1a, 1b, or 1.1-1.37, wherein the composition consists essentially of a mixture of 2-((3,5-bis(trifluoromethyl)phenyl)carbamoyl)-4-chlorophenyl dihydrogen phosphate, meglumine, and a meglumine salt of 2((3,5-bis(trifluoromethyl)phenyl)carbamoyl)-4-chlorophenyl dihydrogen phosphate (e.g., 2-{[3,5-bis(trifluoromethyl)phenyl]carbamoyl}-4-chlorophenyl phosphate bis-meglumine salt) and a disachharide (e.g., one or a mixture of sucrose, lactose, or trehalose).
1.40. Any of Compositions 1a, 1b, or 1.1-1.37, wherein the composition consists essentially of 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, meglumine, and a sugar alcohol (e.g., mannitol). For instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and a sugar alcohol (e.g., mannitol). For instance, any of Compositions 1a, 1b, or 1.1-1.37, wherein the composition consists essentially of 2-{[3,5-bis(trifluoromethyl)phenyl]carbamoyl}-4-chlorophenyl phosphate bis-meglumine salt and mannitol. Or, for instance, any of Compositions 1a, 1b, or 1.1-1.37, wherein the composition consists essentially of a mixture of 2-((3,5-bis(trifluoromethyl)phenyl)carbamoyl)-4-chlorophenyl dihydrogen phosphate, meglumine, and a meglumine salt of 2-((3,5-bis(trifluoromethyl)phenyl)carbamoyl)-4-chlorophenyl dihydrogen phosphate (e.g., 2-{[3,5-bis(trifluoromethyl)phenyl]carbamoyl}-4-chlorophenyl phosphate bis-meglumine salt) and a sugar alcohol (e.g., mannitol).
1.41. Any of Compositions 1a, 1b, or 1.1-1.40, wherein the constitution is reconstituted in 180 seconds.
1.42. Any of Compositions 1a, 1b, or 1.1-1.41, wherein before lyophilization and/or after reconstitution each test sample comprises ≤3 small visible foreign particles (e.g., spots, short fibers below 2 mm, and blocks). Small visible foreign particles may be measured with a clarity detector (e.g., YB-2 clarity detector). Visible Foreign Particles Examination (including determining small visible foreign particles) may be done as described in ChP (Pharmacopeia of the People's Republic of China) 2020 Part 4 <0904>.
1.43. Any of Compositions 1a, 1b, or 1.14.42, wherein the composition is made from a weight ratio of Formula I:sucrose:meglumine of 1:5:0.9-1 (e.g., 1:5:0.93). For instance, wherein the composition is made from 100 mg Formula (1), 500 mg sucrose, 80 mg of meglumine, 133 mg of 10% meglumine solution with water for injection (e.g., sterile water for injection).

Also provided is a method of making a pharmaceutical composition (Method 1a) comprising 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine.

Also provided is a method of making a solid lyophilized pharmaceutical composition (Method 1b) comprising a meglumine salt of 2-((3,5-bis(trifluoromethyl)phenyl)carbamoyl)-4-chlorophenyl dihydrogen phosphate (formula (I)):

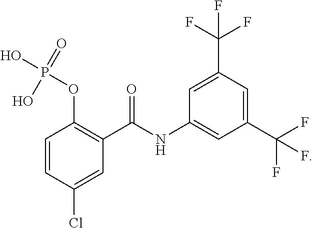 [SMILES: O=C(Nc1cc(C(F)(F)F)cc(C(F)(F)F)c1)c1cc(Cl)ccc1OP(=O)(O)O]

For instance, provided is a method of making any of Compositions 1a, 1b, or 1.1-1.43.

Further provided are Methods 1a and 1b as follows:
1.1. Method 1a or Method 1b, wherein the pharmaceutical composition is any of Compositions 1a, 1b, or 1.1-1.43.
1.2. Method 1a, 1b, or 1.1, wherein 2-((3,5-bis(trifluoromethyl)phenyl)carbamoyl)-4-chlorophenyl dihydrogen phosphate (formula (I)) is in crystalline form, for instance, as described in U.S. Patent Publication No. 2019/0185496, which is hereby incorporated herein by reference in its entirety. For instance, wherein 2-((3,5-bis(trifluoromethyl)phenyl)carbamoyl)-4-chlorophenyl dihydrogen phosphate (formula (I)) is a hydrate crystalline form (e.g., Form N) or a non-solvate, non-hydrate crystalline form (e.g., Form B) as described in U.S. Patent Publication No. 2019/0185496.
1.3. Any of Methods 1a, 1b, 1.1, or 1.2, wherein water (e.g., for injection, e.g., sterile water for injection) is cooled to 15° C.-25° C. (e.g., to 20° C.).
1.4. Method 1.3, wherein meglumine is added to the water (e.g., added to the water and dissolved until clear).
1.5. Method 1.3 or 1.4, wherein 2-((3,5-bis(trifluoromethyl)phenyl)carbamoyl)-4-chlorophenyl dihydrogen phosphate (formula (I)) or a pharmaceutically acceptable salt thereof is added to the water. For instance, Method 1.3 or 1.4, wherein 2-((3,5-bis(trifluoromethyl)phenyl)carbamoyl)-4-chlorophenyl dihydrogen phosphate (formula (I)) is added to the water.
1.6. Any of Methods 1a, 1b, or 1.1-1.5, wherein 2-((3,5-bis(trifluoromethyl)phenyl)carbamoyl)-4-chlorophenyl dihydrogen phosphate (formula (I)) is mixed with meglumine.
1.7. Any of Methods 1a, 1b, or 1.1-1.6, wherein the weight ratio of 2-((3,5-bis(trifluoromethyl)phenyl)carbamoyl)-4-chlorophenyl dihydrogen phosphate (formula (I)) to meglumine is 1:0.2-4, e.g., 1:0.4-2, e.g., 1:0.6-1.
1.8. Any of Methods 1a, 1b, or 1.1-1.7, wherein the molar ratio of 2-((3,5-bis(trifluoromethyl)phenyl)carbamoyl)-4-chlorophenyl dihydrogen phosphate (formula (I)) to meglumine is 1:1-5, e.g., 1:2-5, e.g., 1:2-4, e.g., 1:2-3, e.g., 1:2-2.5, e.g., 1:2-2.3 (e.g., 1:2.1-2.3), e.g., 1:2-2.2, e.g., 1:2.1-2.2, e.g., 1:2.2.
1.9. Any of Methods 1a, 1b, or 1.1-1.8, wherein 2-((3,5-bis(trifluoromethyl)phenyl)carbamoyl)-4-chlorophenyl dihydrogen phosphate is mixed with a lyophilization excipient.
1.10. Any of Methods 1.3-1.9, wherein a lyophilization excipient is added to the water.
1.11. Method 1.9 or 1.10, wherein the lyophilization excipient is selected from one or a mixture of sucrose, lactose, mannitol, glucose, and trehalose, any in free or hydrate form. For instance, Method 1.9 or 1.10, wherein the lyophilization excipient is trehalose, in free or hydrate (e.g., dihydrate) form. For instance, Method 1.9 or 1.10, wherein the lyophilization excipient is mannitol. For instance, Method 1.9 or 1.10, wherein the lyophilization excipient is sucrose. For instance, Method 1.9 or 1.10, wherein the lyophilization excipient is lactose, in free or hydrate (e.g., monohydrate) form.
1.12. Any of Methods 1.9-1.11, wherein the lyophilization excipient is selected from one or a mixture of sucrose, lactose, and trehalose.
1.13. Any of Methods 1.9-1.12, wherein the lyophilization excipient is sucrose.
1.14. Any of Methods 1.9-1.13, wherein the weight ratio of 2-((3,5-bis(trifluoromethyl)phenyl)carbamoyl)-4-chlorophenyl dihydrogen phosphate (formula (I)) to the lyophilization excipient is 1:1-10, e.g., 1:2.5-7.5, e.g., 1:5.
1.15. Any of Methods 1a, 1b, or 1.1-1.14, wherein 2-((3,5-bis(trifluoromethyl)phenyl)carbamoyl)-4-chlorophenyl dihydrogen phosphate is mixed with a pH adjusting agent.
1.16. Any of Methods 1.3-1.15, wherein a pH adjusting agent is added to the water.
1.17. Method 1.15 or 1.16, wherein the pH adjusting agent is selected from one or a mixture of hydrochloric acid, sodium hydroxide, citric acid, and phosphate buffer.
1.18. Any one of Methods 1.15-1.17, wherein the pH adjusting agent is selected from hydrochloric acid and citric acid.
1.19. Any of Methods 1a, 1b, or 1.1-1.18, wherein the pH of the composition (e.g., the aqueous composition) is 7 to 10, e.g., 7.5 to 9.5, e.g., 8 to 9, e.g., 8.2-9, e.g., 8.5-8.6, e.g., 8.5.
1.20. Any of Methods 1a, 1b, or 1.1-1.19, wherein the method comprises filtering (e.g., sterile filter) the mixture of 2-((3,5-bis(trifluoromethyl)phenyl)carbamoyl)-4-chlorophenyl dihydrogen phosphate and meglumine (and optionally lyophilization excipient and optionally pH adjusting agent).
1.21. Any of Methods 1a, 1b, or 1.1-1.20, wherein the method comprises sterilizing the mixture of 2-((3,5-bis(trifluoromethyl)phenyl)carbamoyl)-4-chlorophenyl dihydrogen phosphate and meglumine (and optionally lyophilization excipient and optionally pH adjusting agent).
1.22. Any of Methods 1a, 1b, or 1.1-1.21, wherein the method comprises lyophilizing the mixture of 2-((3,5-bis(trifluoromethyl)phenyl)carbamoyl)-4-chlorophenyl dihydrogen phosphate and meglumine (and optionally lyophilization excipient and optionally pH adjusting agent).
1.23. Method 1.22, wherein the lyophilizing process is as follows:
   (1) hold at −50° C. for 2-6 hours;
   (2) raise temperature to −20° C. to −10° C. and hold for 20-40 hours;
   (3) raise temperature to 20-30° C. (e.g., 25° C.) and hold for 10-30 hours.
1.24. Method 1.22 or 1.23, wherein the lyophilizing process is as follows: (1) decrease temperature to −50° C.; (2) increase temperature to −15° C.; (3) decrease temperature to −50° C.; (4) apply vacuum; (5) decrease temperature to −10° C.; (6) raise temperature to −5° C.; and (7) raise temperature to 25° C. under vacuum.
1.25. Any of Methods 1.22-1.24, wherein the lyophilizing process is as follows: (1) decrease temperature (e.g., shelf temperature) to −40° C. to −60° C. (e.g., −50° C.) within 2-6 hours (e.g., within 4 hours); (2) maintain the temperature (e.g., shelf temperature) at −40° C. to −60° C. (e.g., −50° C.) for 1-2 hours (e.g., 0.5 hours); (3) increase (e.g., rapidly) temperature (e.g., shelf temperature) to −20° C. to −10° C. (e.g., −15° C.); (4) maintain the temperature at −20° C. to −10° C. for 1-3 hours (e.g., 2 hours); (5) decrease (e.g., rapidly) temperature (e.g., slab temperature) to −40° C. to −60° C. (e.g., −50° C.); (6) maintain the temperature (e.g., slab temperature) at −40° C. to −60° C. (e.g., −50° C.) for 2-6 hours (e.g., 4 hours); (7) apply vacuum (e.g., to achieve vacuum below 0.2 mbar); (8) decrease temperature (e.g., shelf temperature) to −20° C. to 0° C. (e.g., −10° C.) within 6-10 hours (e.g., 8 hours); (9) maintain the temperature (e.g., shelf temperature) at −20° C. to 0° C. (e.g., −10° C.) for 8-12 hours (e.g., 10 hours); (10) raise temperature to −10° C. to 0° C. (e.g., −5° C.) within 1-3 hours (e.g., 2 hours); (11) maintain the temperature (e.g., shelf temperature) at −10° C. to 0° C. (e.g., −5° C.) for 13-17 hours (e.g., 15 hours); (12) raise temperature (e.g., shelf temperature) to 20° C.-30° C. (e.g., 25° C.) under vacuum (e.g., ultimate vacuum) within 4-8 hours (e.g., 6 hours); and (13) maintain temperature 20° C.-30° C. (e.g., 25° C.) for 10-14 hours (e.g., 12 hours).
1.26. Any of Methods 1.22-1.25, wherein the lyophilizing process is as follows:
   (1) decrease the shelf temperature to −50° C. within 4 hours and maintain temperature for 0.5 hours;
   (2) increase shelf temperature to −15° C. and maintain temperature for 2 hours;
   (3) decrease slab temperature −50° C. and maintain temperature for 4 hours;
   (4) vacuum pump to achieve a vacuum below 0.2 mbar;
   (5) decrease shelf temperature to −10° C. within 8 hours and maintain temperature for 10 hours;
   (6) raise shelf temperature to −5° C. within 2 hours and maintain temperature for 15 hours; and
   (7) raise shelf temperature to 25° C. within 6 hours under vacuum (e.g., ultimate vacuum) and maintain temperature to dry at 25° C. for 12 hours.
1.27. Any of Methods 1 or 1.1-1.26, wherein the composition is made from a weight ratio of Formula I:sucrose:meglumine of 1:5:0.9-1 (e.g., 1:5:0.93). For instance, wherein the composition before lyophilization is made from 100 mg of Formula (I), 500 mg of sucrose, 80 mg of meglumine, 133 mg of 10% meglumine solution in water, and qs to 10 mL with water for injection (e.g., sterile water for injection).

Also provided is a method (Method 2) of treating or controlling a disease or condition mediated by an aquaporin, e.g., diseases or conditions of water imbalance and other diseases, for example,
edema, for example, edema of the brain or spinal cord, e.g., cerebral edema, e.g. cerebral edema consequent to head trauma, ischemic stroke, glioma, meningitis, acute mountain sickness, epileptic seizure, infection, metabolic disorder, hypoxia (including general systemic hypoxia and hypoxia due to cardiac arrest), water intoxication, hepatic failure, hepatic encephalopathy, diabetic ketoacidosis, abscess, eclampsia, Creutzfeldt-Jakob disease, lupus cerebritis, microgravity and/or radiation exposure, or an invasive central nervous system procedure, e.g., neurosurgery, endovascular clot removal, spinal tap, aneurysm repair, or deep brain stimulation or, e.g., spinal cord edema consequent to spinal cord trauma, e.g., spinal cord compression; or optic nerve edema, e.g., optic nerve edema consequent to microgravity and/or radiation exposure; or
retinal edema; or
pulmonary edema; or
hyponatremia or excessive fluid retention, e.g., consequent to heart failure (HF), liver cirrhosis, nephrotic disorder, syndrome of inappropriate antidiuretic hormone secretion (SIADH), or infertility treatment; or
ovarian hyperstimulation syndrome; or
epilepsy, retinal ischemia or other diseases of the eye associated with abnormalities in intraocular pressure and/or tissue hydration, myocardial ischemia, myocardial ischemia/reperfusion injury, myocardial infarction, myocardial hypoxia, congestive heart failure, sepsis, neuromyelitis optica, or glioblastoma; or
fibromyalgia; or
multiple sclerosis; or
migraines; or
treatment or prophylaxis of transplant rejection, inhibiting rejection of transplanted biological material, or control of edema consequent to a transplant; or for the protection of the heart during heart surgery,
   in a patient (e.g., a human) in need thereof, wherein the method comprises administering to the patient a reconstituted pharmaceutical composition comprising 2((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine. For instance, wherein the method comprises administering the patient a reconstituted pharmaceutical composition comprising a pharmaceutically acceptable salt of 2((3,5-bis(trifluoromethyl)phenyl)carbamoyl)-4-chlorophenyl dihydrogen phosphate (formula (I)). For instance, wherein the method comprises administering to the patient a reconstituted solution obtained from any of Compositions 1a, 1b, or 1.1-1.43.

Further provided is Method 2 as follows:
2.1. Method 2, wherein the disease or condition is described in any of U.S. Pat. Nos. 9,994,514, 9,573,885, and 9,949,991 and U.S. Patent Publication No. 2018/0042873.
2.2. Method 2 or 2.1, wherein the disease or condition is cerebral edema.
2.3. Any of Methods 2, 2.1, or 2.2, wherein the disease or condition is cytotoxic (or cellular) cerebral edema.
2.4. Method 2.3, wherein the cytotoxic cerebral edema is consequent to a stroke (e.g., an ischemic stroke), closed head trauma, traumatic brain injury, or hypoxia.
2.5. Method 2.4, wherein the cytotoxic cerebral edema is consequent to an ischemic stroke.
2.6. Method 2.4, wherein the cytotoxic cerebral edema is consequent to hypoxia.
2.7. Method 2.6, wherein the hypoxia is consequent to a stroke, cardiac arrest, suffocation, or other interruption of oxygen supply or blood flow to the brain.
2.8. Method 2 or 2.1, wherein the disease or condition is spinal cord edema. 2.9. Method 2.8, wherein the spinal cord edema is consequent to spinal cord trauma, e.g., spinal cord compression.
2.10. Any of Methods 2 or 2.1-2.9, wherein the reconstituted solution is obtained from any of Compositions 1a, 1b, or 1.1-1.43.

Further provided is a reconstituted pharmaceutical composition comprising 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine for use to treat any of the diseases or conditions discussed herein, for instance, for use in any of the foregoing methods, e.g., for use in any of Methods 2 or 2.1-2.10. For instance, wherein the reconstituted pharmaceutical composition is obtained from any of Compositions 1a, 1b, or 1.1-1.43. For instance, further provided is a reconstituted pharmaceutical composition comprising a pharmaceutically acceptable salt of 2-((3,5-bis(trifluoromethyl)phenyl)carbamoyl)-4-chlorophenyl dihydrogen phosphate (formula (I)) for use to treat any of the diseases or conditions discussed herein, for instance, for use in any of the foregoing methods, e.g., for use in any of Methods 2 or 2.1-2.10. For instance, wherein the reconstituted pharmaceutical composition is obtained from any of Compositions 1a, 1b, or 1.1-1.43.

Further provided is any of Compositions 1a, 1b, or 1.1-1.43 in the manufacture of a medicament, for instance, in the manufacture of a reconstituted pharmaceutical composition comprising 2-((3,5-bis(trifluoromethyl)phenyl)carbamoyl)-4-chlorophenyl dihydrogen phosphate (formula (I)) or a pharmaceutically acceptable salt thereof, or a pharmaceutically acceptable solvate thereof, and meglumine, for use in any of the foregoing methods, e.g., for use in any of Methods 2 or 2.1-2.10. For instance, further provided is any of Compositions 1a, 1b, or 1.1-1.43 in the manufacture of a medicament, for instance, in the manufacture of a reconstituted pharmaceutical composition comprising a pharmaceutically acceptable salt of 2-((3,5-bis(trifluoromethyl)phenyl)carbamoyl)-4-chlorophenyl dihydrogen phosphate (formula (I)), for use in any of the foregoing methods, e.g., for use in any of Methods 2 or 2.1-2.10.

### DETAILED DESCRIPTION OF THE INVENTION

The invention is further described through specific examples hereinafter, but the described examples are used only to illustrate the present invention and not to limit the present invention.

### Example 1 Lyophilized Composition

TABLE 1
Composition of single dose formulations

| | Formulation | | | | |
|---|---|---|---|---|---|
| | F1 | | F2 | F3 | F4 |
| Formula (I) compound | 100 | mg | 100 mg | 100 mg | 100 mg |
| meglumine | / | | about | / | / |
| | | | 92.6 mg | | |
| arginine | / | | / | about | / |
| | | | | 70 mg | |
| lysine | / | | / | / | about |
| | | | | | 92 mg |
| disodium hydrogen | 96 | mg | / | / | / |
| phosphate | | | | | |
| 2 mol/L sodium | about | | / | / | / |
| hydroxide solution | 227 mg | | | | |
| mannitol | 100 | mg | 100 mg | 140 mg | 140 mg |

| | | | | |
|---|---|---|---|---|
| Water for Injection | QS to | QS to | QS to | QS to |
| | 10 ml | 10 ml | 10 ml | 10 ml |
| pH | 8.5 | 8.5 | 8.5 | 8.5 |

Add the prescription amount of meglumine, arginine, lysine, or disodium hydrogen phosphate/sodium hydroxide to 60-70% of the prescription amount of water for injection at a temperature of about 20° C., stir until it is completely dissolved, and add the prescription amount of formula (I) compound, stir until completely dissolved. Add the prescribed amount of mannitol and stir until completely dissolved.

Replenish the water to the prescribed volume, continue to stir for 20 minutes, filter sterilize through a 0.22 μm filter, fill, half stoppering, and lyophilize.

Take the above samples to test the content and related substances, and measure the turbidity value. The test method is as follows:
(1) Test the content and related substances: tested by high performance liquid chromatography (HPLC), using octadecylsilane-bonded silica gel as a filler (Agilent Eclipse plus C18, 150×4.6 mm, 3.5 μm or equivalent chromatographic column); Using 0.01 mol/L ammonium acetate solution as mobile phase A and acetonitrile as mobile phase B for gradient elution.
(2) Clarity: use a turbidity meter for testing (HACH model: TU5200), take the product solution for testing, and compare with the 0.5# standard turbidity solution (turbidity value range is 1.6-2.2 NTU), if turbidity value is less than 0.5# standard solution, then the product solution is a clear solution.
(3) Moisture: using Karl Fischer moisture analyzer, humidity of the environment is controlled to be less than 30%, take 1 vial of this product, and weigh the total weight; immediately pour into the moisture meter for measurement after opening the lid, and then weigh the empty bottle Weight, measure and calculate the moisture.

The results are shown in Table 2.

TABLE 2
stability of lyophilized formulations in table 1

| | | Formula | | Turbidity |
|---|---|---|---|---|
| | Storage | (II) | Total | value |
| Formulation | condition | compound % | impurity % | (NTU) |
| F1 | lyophilization 0 h | 0.89 | 0.89 | 11.3 |
| F2 | lyophilization 0 h | 0.16 | 0.16 | 0.808 |
| | 25° C., 10 d | 0.38 | 0.38 | 0.89 |
| F3 | lyophilization 0 h | 0.38 | 0.38 | 0.969 |
| | 25° C., 10 d | 0.98 | 1.00 | 1.28 |
| F4 | lyophilization 0 h | 0.35 | 0.35 | 0.748 |
| | 25° C., 10 d | 0.90 | 0.90 | 3.29 |

### Example 2 Lyophilized Composition

TABLE 3
Composition of single dose formulations

| | Formulation | | | | | | | |
|---|---|---|---|---|---|---|---|---|
| | F5 | | F6 | | F7 | | F8 | |
| Formula (I) | 100 | mg | 100 | mg | 100 | mg | 100 | mg |
| compound | | | | | | | | |
| mannitol | 140 | mg | 100 | mg | 100 | mg | 100 | mg |
| meglumine | 42.7 | mg | 99.0 | mg | 132.6 | mg | 186.5 | mg |

| | | | | |
|---|---|---|---|---|
| Water for | QS to 10 ml | QS to 10 ml | QS to 10 ml | QS to 10 ml |
| Injection | | | | |
| pH | 7.5 | 9.0 | 9.5 | 9.5 |

Add prescription amount of meglumine to 60-70% of the prescription amount of water for injection at a temperature of about 20° C., stir until it is completely dissolved, add the prescription amount of formula (I) compound, and stir until completely dissolved.

Replenish the water to the prescribed volume, continue to stir for 20 minutes, filter sterilize through a 0.22 μm filter, fill, half stoppering, and lyophilize.

Take the above samples to test the content and related substances, and measure the turbidity value. The results are shown in Table 4.

TABLE 4
stability of lyophilized formulations in table 3

| | | | | | Turbidity |
|---|---|---|---|---|---|
| Formu- | Storage | | Formula (II) | Total | value |
| lation | condition | pH | compound % | impurity % | (NTU) |
| F5 | lyophilization | 7.5 | 0.10 | 0.16 | 15.7 |
| | 0 h | | | | |
| F2 | lyophilization | 8.5 | 0.16 | 0.16 | 0.808 |
| | 0 h | | | | |
| | 25° C., 10 d | 8.5 | 0.38 | 0.38 | 0.89 |
| | 25° C., 1 M | 8.6 | 0.81 | 0.81 | 0.511 |
| | 2~8° C., 1 M | 8.6 | 0.18 | 0.18 | 0.65 |
| | −20° C., 1 M | 8.6 | 0.13 | 0.13 | 0.768 |
| F6 | lyophilization | 9.0 | 0.22 | 0.22 | 0.691 |
| | 0 h | | | | |
| | 25° C., 10 d | 9.0 | 0.42 | 0.46 | 0.391 |
| | 25° C., 1 M | 9.0 | 0.92 | 0.92 | 0.527 |
| | 2~8° C., 1 M | 9.1 | 0.15 | 0.15 | 0.432 |
| | −20° C., 1 M | 9.0 | 0.12 | 0.12 | 0.771 |
| F7 | lyophilization | 9.5 | 0.12 | 0.15 | 0.863 |
| | 0 h | | | | |
| | 25° C., 10 d | 9.6 | 0.95 | 0.98 | 0.829 |
| | 25° C., 1 M | 9.5 | 2.79 | 2.79 | 0.699 |
| | 2~8° C., 1 M | 9.6 | 0.17 | 0.17 | 0.460 |
| | −20° C., 1 M | 9.6 | 0.19 | 0.19 | 0.461 |
| F8 | lyophilization | 10.0 | 0.27 | 0.27 | 0.321 |
| | 0 h | | | | |
| | 25° C., 10 d | 10.1 | 3.52 | 3.52 | 0.593 |
| | 25° C., 1 M | 9.9 | 9.67 | 9.67 | 0.714 |
| | 2~8° C., 1 M | 9.9 | 0.33 | 0.33 | 0.659 |
| | −20° C., 1 M | 9.9 | 0.43 | 0.44 | 0.53 |

### Example 3 Lyophilized Composition

TABLE 5
Composition of single dose formulations

| | | Formulation | | | | |
|---|---|---|---|---|---|---|
| | | F9 | F10 | F11 | F12 | F13 |
| Formula (I) compound | | 100 mg | 100 mg | 100 mg | 100 mg | 100 mg |
| meglumine | | about | about | about | about | about |
| | | 95.0 mg | 92.9 mg | 93.3 mg | 95 mg | 90 mg |
| lyophilization | mannitol | 250 mg | / | / | / | / |
| excipient | Trehalose dihydrate | / | 500 mg | / | / | / |
| | sucrose | / | / | 500 mg | / | / |
| | lactose | / | / | / | 500 mg | / |
| | Sulfobutylether-β- | / | / | / | / | 500 mg |
| | cyclodextrin sodium | | | | | |

| | | | | | |
|---|---|---|---|---|---|
| Water for Injection | QS to | QS to | QS to | QS to | QS to |
| | 10 ml | 10 ml | 10 ml | 10 ml | 10 ml |
| pH | 8.5~9 | 8.5~9 | 8.5~9 | 8.5~9 | 8.5~9 |

Add the prescription amount of meglumine to 70-80% of the prescription amount of water for injection at a temperature of about 20° C., stir until it is completely dissolved, add the prescription amount of formula (I) compound, stir until completely dissolved, and then add the prescription amount of lyophilized excipient and stir until completely dissolved.

Replenish the water to the prescribed volume, continue to stir for 20 minutes, filter sterilize through a 0.22 μm filter, fill, half stoppering, and lyophilize.

Take the above samples to test the content and related substances, and measure the turbidity value. The results are shown in Table 6.

TABLE 6
stability of lyophilized formulations in table 5

| | | | Content of | | Turbidity |
|---|---|---|---|---|---|
| Formu- | Storage | | Formula (II) | Total | value |
| lation | condition | pH | compound % | impurity % | (NTU) |
| F9 | intermediate | 9.0 | 0.07 | 0.09 | 0.167 |
| | (solution before | | | | |
| | lyophilization) | | | | |
| | lyophilization | 8.9 | 0.13 | 0.13 | 0.518 |
| | 0 h | | | | |
| | 25° C., 10 d | 8.8 | 0.52 | 0.52 | 0.361 |
| | 25° C., 23 d | 8.9 | 1.05 | 1.05 | 0.576 |
| | 2~8° C., 1 M | 8.9 | 0.17 | 0.17 | clear |
| | −20° C., 1 M | 8.8 | 0.11 | 0.11 | clear |
| F10 | intermediate | 8.9 | 0.05 | 0.07 | 0.176 |
| | (solution before | | | | |
| | lyophilization) | | | | |
| | lyophilization | 8.8 | 0.12 | 0.12 | 0.317 |
| | 0 h | | | | |
| | 25° C., 10 d | 8.8 | 0.31 | 0.31 | 0.466 |
| | 25° C., 23 d | 8.8 | 0.55 | 0.97 | 0.465 |
| | 2~8° C., 1 M | 8.7 | 0.15 | 0.15 | 0.396 |
| | −20° C., 1 M | 8.7 | 0.10 | 0.10 | 0.491 |
| F11 | intermediate | 9.0 | 0.05 | 0.08 | 0.165 |
| | (solution before | | | | |
| | lyophilization) | | | | |
| | lyophilization | 8.8 | 0.09 | 0.09 | 0.608 |
| | 0 h | | | | |
| | 25° C., 10 d | 8.8 | 0.15 | 0.15 | 0.541 |
| | 25° C., 23 d | 8.8 | 0.31 | 1.00 | 0.474 |
| | 2~8° C., 1 M | 8.8 | 0.10 | 0.10 | 0.622 |
| | −20° C., 1 M | 8.7 | 0.08 | 0.08 | 0.533 |
| F12 | intermediate | 9.0 | 0.05 | 0.07 | 0.183 |
| | (solution before | | | | |
| | lyophilization) | | | | |
| | lyophilization | 8.8 | 0.13 | 0.13 | 0.599 |
| | 0 h | | | | |
| | 25° C., 10 d | 8.8 | 0.25 | 0.25 | 0.352 |
| | 25° C., 23 d | 8.8 | 0.46 | 0.48 | 0.564 |
| | 2~8° C., 1 M | 8.8 | 0.14 | 0.14 | 0.375 |
| | −20° C., 1 M | 8.8 | 0.10 | 0.10 | 0.548 |
| F13 | intermediate | 8.6 | 0.07 | 0.11 | 0.699 |
| | (solution before | | | | |
| | lyophilization) | | | | |
| | lyophilization | 8.4 | 0.33 | 0.33 | 1.420 |
| | 0 h | | | | |
| | 25° C., 10 d | 8.4 | 1.64 | 1.88 | 0.582 |
| | 25° C., 1 M | 8.5 | 2.72 | 3.02 | 0.550 |
| | 2~8° C., 1 M | 8.6 | 0.71 | 0.80 | 0.592 |
| | −20° C., 1 M | 8.5 | 0.42 | 0.56 | 0.626 |

### Example 4 Lyophilized Composition

TABLE 7
Composition of single dose formulations

| composition | Prescription amount/vial | |
|---|---|---|
| Formula (I) compound | 100 | mg |
| sucrose | 500 | mg |
| meglumine | 80 | mg |
| 10% meglumine solution | 133 | mg |

| | |
|---|---|
| Water for injection | QS to 10 ml |

Add the prescription amount of meglumine to 80% of the prescription amount of water for injection at a temperature of about 20° C., stir until it is completely dissolved, add the prescription amount of formula (I) compound, stir until the dissolution is complete, then add the prescription amount of sucrose, and stir until completely dissolved.

Adjust the pH of the above solution to 8.5-9 with 10% meglumine solution, replenish water to the prescribed volume, continue to stir for 20 minutes, filter sterilize through a 0.2 μm filter, fill, half stoppering, and lyophilize.

### Lyophilization Process:

Pre-lyophilizing: decrease the shelf temperature to −50° C. within 4 h, and maintain the temperature for 0.5 h, then rapidly increase the shelf temperature to −15° C., and maintain the temperature for 2 h, then rapidly decrease the slab temperature to −50° C., and maintain the temperature for 4 h to achieve a completely frozen product. Turn on the vacuum pump to achieve a vacuum below 0.2 mbar, and the sublimation starts.

Sublimation stage: decrease the shelf temperature to −10° C. within 8 h and maintain the temperature for 10 h, then raise the shelf temperature to −5° C. within 2 h and maintain the temperature for about 15 h.

Secondary drying: raise the shelf temperature to 25° C. within 6 h under ultimate vacuum, and maintain the temperature to dry at 25° C. for about 12 h.

Take the above samples to test the content and related substances, and measure the turbidity value. The results are shown in Table 8.

TABLE 8
stability of lyophilized formulation in table 7

| | Content of | | Turbidity |
|---|---|---|---|
| Storage | Formula (II) | Total | value |
| conditions | compound % | impurity % | (NTU) |
| intermediate | 0.05 | 0.08 | 0.165 |
| (solution before | | | |
| lyophilization) | | | |
| lyophilization, 0 h | 0.09 | 0.09 | 0.608 |
| 25° C., 10 d | 0.15 | 0.15 | 0.541 |
| 25° C., 23 d | 0.31 | 1.00 | 0.474 |
| 2~8° C., 1 M | 0.10 | 0.10 | 0.622 |
| −20° C., 1 M | 0.08 | 0.08 | 0.533 |

### Example 5

TABLE 9

| Composition | Amount/vial | | | |
|---|---|---|---|---|
| Formula (I) compound | 100 | mg | 100 | mg |
| meglumine | 80 | mg | 80 | mg |

| | | | |
|---|---|---|---|
| sucrose | 500 | mg | / |

| | | | |
|---|---|---|---|
| hydroxypropyl-beta-cyclodextrin | / | 1 | g |

| | | |
|---|---|---|
| (HP-β-CD) | | |
| meglumine | Adjust pH to 8.5 | Adjust pH to 8.0 |
| Water for injection | QS to 10 ml | QS to 10 ml |

TABLE 10
Stability comparison of HP-β-CD and sucrose formulations

| | | Sucrose formulation | | HP-β-CD formulation |
|---|---|---|---|---|
| Stability | | Content of Formula (II) | | Content of Formula (II) |
| Conditions | | compound % | | compound % |
| 0 | h | 0.025% | | 0.09% |
| 25° C. ± 2° C. | | 0.47% | (6 months) | 2.89% (4 months) |
| 2~8° | C. | 0.072% | (6 months) | 0.46% (4 months) |

| | | | |
|---|---|---|---|
| −20° C. ± 5° C. | 0.040% | (6 months) | 0.12% (4 months) |

### Example 6 Lyophilized Composition

TABLE 11
Composition of single dose formulations

| composition | Prescription amount/vial | |
|---|---|---|
| Formula (I) compound | 100 | mg |
| sucrose | 500 | mg |
| meglumine | 80 | mg |
| 10% meglumine solution | 133 | mg |

| | |
|---|---|
| Water for injection | QS to 10 ml |

TABLE 12
stability of lyophilized formulation in table 11

| | Content of | | |
|---|---|---|---|
| | Formula (II) | Total | Water |
| Storage conditions | compound % | impurity % | (Karl Fisher) |
| 0 month | 0.025% | 0.025% | 0.8% |
| 25° C. ± 2° C., 6 months | 0.50% | 0.50% | 1.3% |
| 2~8° C., 6 months | 0.088% | 0.088% | 1.3% |
| −20° ± 5° C., 6 months | 0.041% | 0.041% | 1.1% |

### Example 7

TABLE 13

| | | Content of | | | |
|---|---|---|---|---|---|
| Formulation | | Formula (II) | Total | Clarity | |
| Composition | Stability Data | compound % | Impurities % | (NTU*) | pH |
| Formula (I) compound + | 0 h | 0.04 | 0.07 | N/A | N/A |
| meglumine + sucrose | | | | | |
| 10 mg/ml | | | | | |
| Formula (I) compound + | 0 h | 0.03 | 0.07 | N/A | N/A |
| meglumine + trehalose | | | | | |
| 10 mg/ml | | | | | |
| Formula (I) compound + | 0 h | 0.04 | 0.09 | N/A | N/A |
| meglumine + lactose | | | | | |
| 10 mg/ml | | | | | |
| Formula (I) compound + | Solution before | 0.07 | 0.12 | Solution clear | 8.6 |
| sodium hydroxide | lyophilization | | | 0.668 NTU | |
| 10 mg/ml | 0 h | 0.55 | 0.63 | Solution turbid | 7.8 |
| | | | | 10.9 NTU | |
| Formula (I) compound + | Solution before | 0.11 | 0.16 | Solution clear | 8.5 |
| sodium hydroxide + | lyophilization | | | 0.674 NTU | |
| sulfobutylether-β- | 0 h | 0.98 | 1.01 | Solution clear | 7.5 |
| cyclodextrin | | | | 0.712 NTU | |

*0.5 standard turbidity solution is 1.6-2.2 NTU and solutions below this turbidity value are clear solutions.

### Example 8

TABLE 14

| | | Content of | | | |
|---|---|---|---|---|---|
| Formulation | | Formula (II) | Total | Clarity | |
| Composition | Stability Data | compound % | Impurities % | (NTU*) | pH |
| Formula (I) + histidine + | Solution before | 0.05 | 0.07 | Solution clear | 6.6 |
| mannitol 10 mg/ml | lyophilization | | | | |
| | 0 h | 0.08 | 0.08 | Solution clear | 6.7 |
| | | | | 1.85 NTU | |
| | 25° C., 10 days | 0.24 | 0.30 | Solution turbid | 6.6 |
| | | | | 13.7 NTU | |
| Formula (I) + Arginine + | Solution before | 0.07 | 0.07 | Solution clear | / |
| mannitol 10 mg/ml | lyophilization | | | | |
| | 0 h | 0.09 | 0.23 | Solution clear | 6.5 |
| | | | | 1.08 NTU | |
| | 25° C., 10 days | 0.55 | 0.59 | Solution turbid | 6.6 |
| | | | | 55.9 NTU | |
| Formula (I) + Arginine + | Solution before | 0.30 | 0.30 | Solution clear | 8.5 |
| mannitol 10 mg/ml | lyophilization | | | 0.634 NTU | |
| | 0 h | 0.38 | 0.38 | Solution clear | 8.6 |
| | | | | 0.969 NTF | |
| | 25° C., 10 days | 0.98 | 1.00 | Solution clear | 8.5 |
| | | | | 1.28 NTU | |
| Formula (I) + Lysine + | Solution before | 0.14 | 0.16 | Solution clear | 7.7 |
| mannitol 10 mg/ml | lyophilization | | | | |
| | 0 h | 0.21 | 0.21 | Solution clear | 7.8 |
| | | | | 0.842 NTU | |
| | 25° C., 10 days | 0.81 | 0.85 | Solution turbid | 7.7 |
| | | | | 71.8 NTU | |
| Formula (I) + Lysine + | Solution before | 0.31 | 0.33 | Solution clear | 8.5 |
| mannitol 10 mg/ml | lyophilization | | | 0.696 NTU | |
| | 0 h | 0.35 | 0.35 | Solution clear | 8.5 |
| | | | | 0.748 NTU | |
| | 25° C., 10 days | 0.90 | 0.90 | Solution turbid | 8.4 |
| | | | | 3.29 NTU | |
| Formula (I) + | Solution before | 0.33 | 0.33 | Solution clear | 6.4 |
| phosphate + 40% | lyophilization | | | 1.68 NTU | |
| tertiary butyl | 0 h | 0.41 | 0.41 | Solution turbid | 5.1 |
| alcohol + mannitol | | | | 14.3 NTU | |
| 10 mg/ml | | | | | |
| Formula (I) + | Solution before | 0.35 | 0.38 | Solution clear | 6.5 |
| phosphate + 20% | lyophilization | | | 0.943 NTU | |
| tertiary butyl | 0 h | 0.45 | 0.45 | Solution turbid | 6.0 |
| alcohol + mannitol | | | | 42.3 NTU | |
| 10 mg/ml | | | | | |
| Formula (I) + | Solution before | 0.25 | 0.29 | Solution turbid | 6.4 |
| phosphate + 10% | lyophilization | | | 5.92 NTU | |
| tertiary butyl | 0 h | 0.33 | 0.33 | Solution turbid | 6.3 |
| alcohol + mannitol | | | | 20.8 NTU | |
| 10 mg/ml | | | | | |
| Formula (I) + | Solution before | 0.33 | 0.33 | Solution clear | 7.0 |
| meglumine + 20% | lyophilization | | | 2.03 NTU | |
| tertiary butyl | 0 h | 0.29 | 0.29 | Solution turbid | 6.1 |
| alcohol + mannitol | | | | 15.4 NTU | |
| 10 mg/ml | | | | | |
| Formula (I) + | Solution before | 0.06 | 0.06 | Solution clear | 8.9 |
| meglumine ++ mannitol | lyophilization | | | 0.233 NTU | |
| 10 mg/ml | 0 h | 0.08 | 0.08 | Solution clear | 8.8 |
| | | | | 0.436 NTU | |
| | 25° C., 10 days | 0.44 | 0.44 | Solution clear | 8.8 |
| | | | | 0.418 NTU | |

*0.5 standard turbidity solution is 1.6-2.2 NTU and solutions below this turbidity value are clear solutions.

Lyophilized preparations with histidine, arginine, and lysine are turbid after 10 days at 25° C. With tertiary butyl alcohol, samples are turbid after lyophilization and pH decreases after reconstitution.

### Example 9

### Reconstitution Solvents

TABLE 15

| Solvent | Results |
|---|---|
| Water for injection | Not conform to the ChP |
| Glucose injection | Similar to water resolution |
| Fructose sodium diphosphate | Insoluble, jelly-like |
| injection | |
| 0.9% sodium chloride injection | Conform to the ChP |
| Potassium chloride injection | 3 batches of samples |
| | (1 bottle has ≥3 cilia) |
| Ringer's solution of | Similar to the 0.9% sodium |
| sodium lactate | chloride injection |
| Ringer's solution of | Similar to the 0.9% sodium |
| sodium acetate | chloride injection |

1. A pharmaceutical composition comprising 2-((3,5-bis(trifluoromethyl)phenyl)carbamoyl)-4-chlorophenyl dihydrogen phosphate or pharmaceutically acceptable salt thereof, or a pharmaceutically acceptable solvate thereof, and meglumine.

2. The pharmaceutical composition of claim 1, wherein the weight ratio of 2-((3,5-bis(trifluoromethyl)phenyl)carbamoyl)-4-chlorophenyl dihydrogen phosphate to meglumine is 1:0.2˜4.

3. The pharmaceutical composition of claim 2, wherein the weight ratio of 2-((3,5-bis(trifluoromethyl)phenyl)carbamoyl)-4-chlorophenyl dihydrogen phosphate to meglumine is 1:0.4˜2.

4. The pharmaceutical composition of claim 3, wherein the weight ratio of 2-((3,5-bis(trifluoromethyl)phenyl)carbamoyl)-4-chlorophenyl dihydrogen phosphate to meglumine is 1:0.6˜1.

5. The pharmaceutical composition of claim 1, wherein the pharmaceutical composition is an injectable pharmaceutical composition, preferably a lyophilized pharmaceutical composition.

6. The pharmaceutical composition of claim 1, which further comprises a lyophilization excipient.

7. The pharmaceutical composition of claim 6, wherein the lyophilization excipient is selected from one or a mixture of sucrose, lactose, mannitol, glucose, and trehalose.

8. The pharmaceutical composition of claim 7, wherein the lyophilization excipient is selected from one or a mixture of sucrose, lactose, and trehalose.

9. The pharmaceutical composition of claim 6, wherein the weight ratio of 2-((3,5-bis(trifluoromethyl)phenyl)carbamoyl)-4-chlorophenyl dihydrogen phosphate to lyophilization excipient is 1:1-10, preferably 1:2.5-7.5, more preferably 1:5.

10. The pharmaceutical composition of claim 1, which further comprises a pH adjusting agent.

11. The pharmaceutical composition of claim 10, wherein the pH adjusting agent is selected from one or a mixture of hydrochloric acid, sodium hydroxide, citric acid, and phosphate buffer, preferably selected from hydrochloric acid or citric acid.

12. The pharmaceutical composition of claim 1, wherein the pH is 7.5 to 9.5, preferably the pH is 8.0 to 9.0, and more preferably the pH is about 8.5.

13. A pharmaceutical composition comprising 2-((3,5-bis(trifluoromethyl)phenyl)carbamoyl)-4-chlorophenyl dihydrogen phosphate or pharmaceutically acceptable salt thereof, or a pharmaceutically acceptable solvate thereof, meglumine, a lyophilization excipient and pH adjusting agent.

14. The pharmaceutical composition of claim 13, wherein the pharmaceutical composition comprises ≤1% of N-[3,5-bis(trifluoromethyl)phenyl]-5-chloro-2-hydroxybenzamide after 6 months at room temperature.

15. The pharmaceutical composition of claim 1, wherein the composition has 3% w/w water.

16. A method for preparing the pharmaceutical composition according to claim 1, comprising:
a) Taking 60%-90% of the prescription amount of water for injection, cooling to 15° C.-25° C., adding meglumine and dissolving until clear, slowly adding 2-((3,5-bis(trifluoromethyl)phenyl)carbamoyl)-4-chlorophenyl dihydrogen phosphate or a pharmaceutically acceptable salt thereof, or a pharmaceutically acceptable solvate thereof;
b) Optionally, adding other components;
c) Adding prescribed amount of water for injection;
d) Optionally, sterile filtering and lyophilizing the product obtained in step (c).

17. The method of claim 16, wherein the step (a) is cooled to 20° C.

18. The method of claim 16, wherein the other components in step (b) are selected from lyophilization excipients and pH adjusting agents.

19. The method of claim 16, wherein the lyophilizing process in step (d) is as follows: (1) preserve at −50° C. for 2-6 h; (2) raise temperature to −20° C. to −10° C. and preserve for 20-40 h; (3) raise temperature to 20-30° C. and preserve for 10-30 h.

20. The method of claim 19, wherein the temperature is raised to 25° C. in step (3).
